(19) United States
(12) Patent Application Publication
(10) Pub. No.: US 2021/0065884 A1
(43) Pub. Date: Mar. 4, 2021

(54) SYSTEM FOR RECORDING ATTENDANCE OF CAREGIVER

(71) Applicant: Shih-Chang CHAO, Kaohsiung City (TW)

(72) Inventor: Shih-Chang CHAO, Kaohsiung City (TW)

(21) Appl. No.: 16/560,618

(22) Filed: Sep. 4, 2019

(51) Int. Cl. G16H 40/20; G06Q 10/10

(52) U.S. Cl. CPC G16H 40/20; G06Q 10/1091

(57) ABSTRACT

A system includes a client terminal, a caregiver terminal and a server communicable with the client terminal and the caregiver terminal. The server stores a transaction record recording a caregiving period for a care service and containing a state of an attendance switch. The client terminal is operable to designate the state of the attendance switch. The caregiver terminal includes an output unit and a processor connected to each other. The processor determines whether a current time is around the start of the caregiving period, and controls the output unit to provide an attendance interface for a caregiver to sign in or out with respect to the care service upon arrival or departure when the determination is affirmative.

### FIELD

The disclosure relates to a system of care service, and more particularly to a system for recording attendance of a caregiver.

### BACKGROUND

Conventionally, attendance of a caregiver is not precisely recorded. Consequently, the recorded/presumed hours of care service may be inconsistent with the actual hours of service provided by the caregiver, resulting in unfair transaction between the caregiver and the client that hires the caregiver for the care service.

### SUMMARY

Therefore, an object of the disclosure is to provide a system for recording attendance of a caregiver that can alleviate at least one of the drawbacks of the prior art.

According to the disclosure, the caregiver is expected to provide care service at a location within a caregiving period according to an agreement on the care service made between the caregiver and a client. The system includes a server, a client terminal and a caregiver terminal.

The server is configured to store a transaction record that is related to the agreement, that records the caregiving period and an address corresponding to the location, and that contains a state of an attendance switch.

The client terminal is possessed by the client, is capable of communicating with the server, and is configured to be operated to designate the state of the attendance switch which is contained in the transaction record stored on the server.

The caregiver terminal is possessed by the caregiver, is capable of communicating with the server, and includes an output unit and a processor. The processor is electrically connected to the output unit and is configured to determine whether a current time is around the start of the caregiving period, and to control the output unit to provide an attendance interface for the caregiver to sign in or out with respect to the care service upon arrival or departure when it is determined that the current time is around the start of the caregiving period. The attendance interface is related to the state of the attendance switch.

### BRIEF DESCRIPTION OF THE DRAWINGS

Other features and advantages of the disclosure will become apparent in the following detailed description of the embodiment with reference to the accompanying drawings, of which:

FIG. 1 is a block diagram illustrating an embodiment of a system for recording attendance of a caregiver according to the disclosure;

FIG. 2 is a block diagram illustrating an embodiment of a client terminal of the system according to the disclosure;

FIG. 3 is a block diagram illustrating an embodiment of a caregiver terminal of the system according to the disclosure;

FIG. 4 is a schematic diagram illustrating an embodiment of an appointment interface provided by the client terminal according to the disclosure;

FIG. 5 is a schematic diagram illustrating an embodiment of a quotation interface provided by the caregiver terminal according to the disclosure;

FIG. 6 is a schematic diagram illustrating an embodiment of a payment interface provided by the client terminal according to the disclosure;

FIG. 7 is a schematic diagram illustrating an embodiment of a transaction record to be presented by the client terminal or the caregiver terminal according to the disclosure;

FIG. 8 is a schematic diagram illustrating an embodiment of a switch-setting interface provided by the client terminal according to the disclosure;

FIG. 9 is a schematic diagram illustrating an embodiment of an attendance interface provided by the caregiver terminal according to the disclosure;

FIG. 10 is a schematic diagram illustrating an embodiment of an attendance-checking interface provided by the client terminal according to the disclosure;

FIG. 11 is a schematic diagram illustrating an embodiment of another attendance-checking interface provided by the caregiver terminal according to the disclosure;

FIG. 12 is a schematic diagram illustrating an embodiment of an attendance-questioning interface provided by the client terminal according to the disclosure; and

FIG. 13 is a schematic diagram illustrating an embodiment of another attendance-questioning interface provided by the caregiver terminal according to the disclosure.

### DETAILED DESCRIPTION

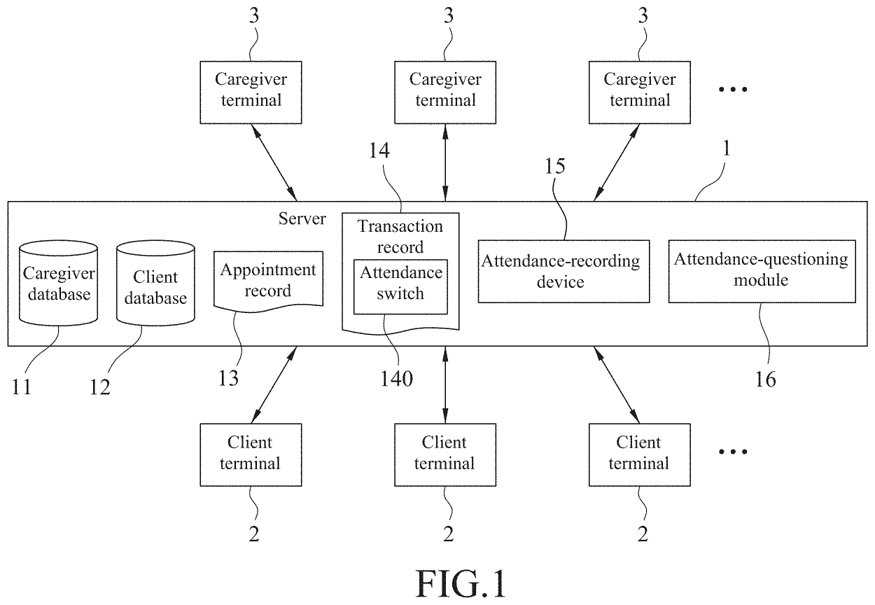

Referring to FIGS. 1 to 3, an embodiment of a system for recording attendance of a caregiver according to this disclosure is illustrated. The caregiver is expected to provide care service to a care receiver at a location within a caregiving period according to an agreement on the care service made between the caregiver and a client. The care service may be a long-term care service, babysitting service, or the like. The caregiver may be a caretaker or a babysitter. The client may be related to a person who demands and is going to receive the care service (i.e., the care receiver). For example, the client and the care receiver are family.

The system includes a server 1, a plurality of client terminals 2 and a plurality of caregiver terminals 3 as shown in FIG. 1. However, for the sake of clear explanation, only one client terminal 2 and one caregiver terminal 3 will be discussed in the following embodiment.

Referring to FIG. 1, the server 1 includes a caregiver database 11, a client database 12, an attendance-recording device 15 and an attendance-questioning module 16. The server 1 is configured to store an appointment record 13 that is related to an appointment for the care service requested by the client, and a transaction record 14 that is related to the agreement on the care service. The transaction record 14 records the caregiving period and an address corresponding to the location where the care service is to be provided, and contains a state of an attendance switch 140. The attendance-recording device 15 may be implemented to be flash memory, a hard disk drive (HDD), a solid state disk (SSD), electrically-erasable programmable read-only memory (EEPROM) or any other non-volatile memory devices, but is not limited thereto.

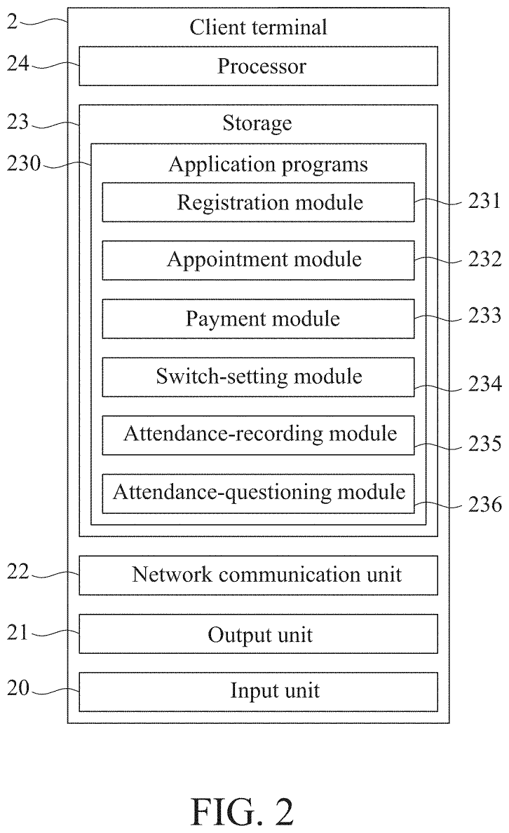

Referring to FIG. 2, the client terminal 2 is possessed by the client, and is capable of communicating with the server 1. The client terminal 2 is configured to be operated to designate the state of the attendance switch 140, and the state of the attendance switch 140 is contained in the transaction record 14 stored on the server 1 by use of, for example, a bit field, a flag, or the like. In this embodiment, the client terminal may be a smartphone or a tablet computer, but implementation of the client terminal 2 is not limited to the disclosure herein and may vary in other embodiments. The client terminal 2 includes an input unit 20, an output unit 21, a network communication unit 22, a storage 23, and a processor 24 that is electrically connected to the input unit 20, the output unit 21, the network communication unit 22 and the storage 23.

In this embodiment, the input unit 20 of the client terminal 2 is implemented to be a keyboard, and the output unit 21 of the client terminal 2 is implemented to be a display device. However, implementations of the input unit 20 and the output unit 21 of the client terminal 2 are not limited to the disclosure herein and may vary in other embodiments. For example, in some embodiments, the input unit 20 and the output unit 21 may be integrated as a touchscreen.

The network communication unit 22 of the client terminal 2 is implemented to be a network interface controller or a wireless transceiver that supports wired/wireless communication standards, but is not limited thereto.

The storage 23 of the client terminal 2 is implemented to be flash memory, a HDD, an SSD, an EEPROM or any other non-volatile memory devices, but is not limited thereto. The storage 23 is configured to store an application program (app) 230. In this embodiment, the app 230 includes a registration module 231 that enables a person to register to become a client of the care service, an appointment module 232 that enables the client to make an appointment for the care service, a payment module 233 that enables the client to make payment for the care service, a switch-setting module 234 that enables the client to designate the state of the attendance switch 140, an attendance-recording module 235 that enables the client to check attendance history of the caregiver, and an attendance-questioning module 236 that enables the client to question (cast doubt on) the attendance history of the caregiver.

The processor 24 of the client terminal 2 is implemented to be a central processing unit (CPU), a microprocessor, a micro control unit (MCU), or any circuit configurable/programmable in a software manner and/or hardware manner to implement functionalities disclosed in this disclosure in relation to the processor 24.

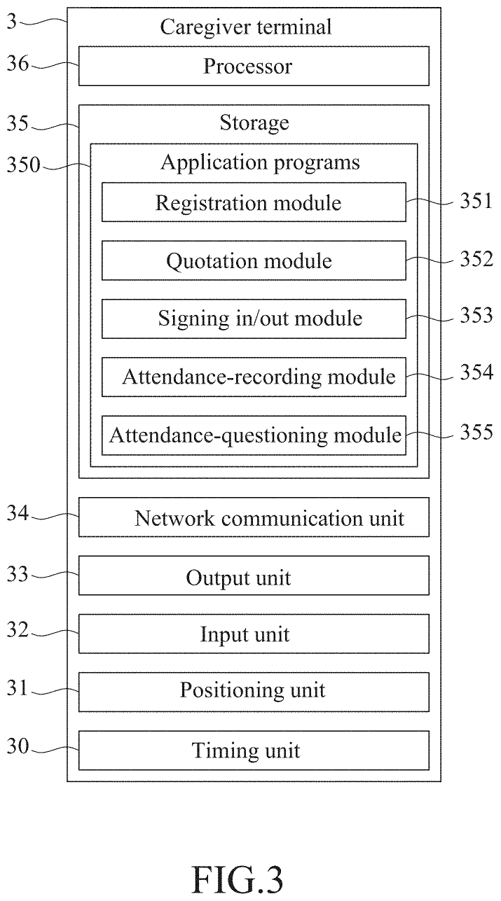

Referring to FIG. 3, the caregiver terminal 3 is possessed by the caregiver, and is capable of communicating with the server 1. Similar to the client terminal 2, in this embodiment, the caregiver terminal 3 may be a smartphone or a tablet computer, but implementation of the caregiver terminal 3 is not limited to the disclosure herein and may vary in other embodiments.

The caregiver terminal 3 includes a timing unit 30, a positioning unit 31, an input unit 32, an output unit 33, a network communication unit 34, a storage 35, and a processor 36 that is electrically connected to the timing unit 30, the positioning unit 31, the input unit 32, the output unit 33, the network communication unit 34 and the storage 35. Implementation of the caregiver terminal 3 is similar to that of the client terminal 2, and the input unit 32, the output unit 33, the network communication unit 34, the storage 35 and the processor of the caregiver terminal 3 are respective counterparts of the input unit 20, the output unit 21, the network communication unit 22, the storage 23 and the processor 24 of the client terminal 2. Consequently, only the differences between the caregiver terminal 3 and the client terminal 2 are discussed below.

The timing unit 30 of the caregiver terminal 3 is configured to determine a current time, and to transmit information regarding the current time to the processor 36. In this embodiment, the timing unit 30 is implemented to be a timer or a clock, but implementation of the timing unit 30 is not limited to the disclosure herein and may vary in other embodiments.

The positioning unit 31 of the caregiver terminal 3 is configured to detect a position of the caregiver terminal 3 and to transmit information regarding the position of the caregiver terminal 3 to the server 1 via the network communication unit 34. In this embodiment, the positioning unit 31 is implemented to be a Global Positioning System (GPS) device, but implementation of the positioning unit 31 is not limited to the disclosure herein and may vary in other embodiments.

The storage 35 of the caregiver terminal 3 is configured to store another application program (app) 350. In this embodiment, said another app 350 includes a registration module 351 that enables a person to register to become a caregiver of the care service, a quotation module 352 that enables the caregiver to submit to the server 1 a cost estimate for the care service requested by the client, a signing in/out module 353 that enables the caregiver to sign in or out with respect to the care service upon arrival or departure, an attendance-recording module 354 that enables the caregiver to check his/her attendance history, and an attendance-questioning module 355 that enables the caregiver to question (cast doubt on) the attendance history of himself/herself.

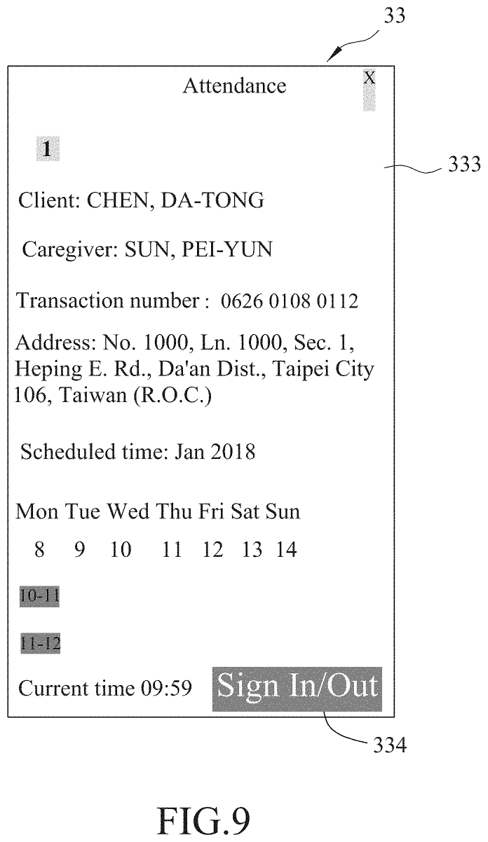

The processor 36 of the caregiver terminal 3 is configured to determine whether the current time is around the start of the caregiving period, and to control the output unit 33 to provide an attendance interface 333 (see FIG. 9) when it is determined that the current time is around the start of the caregiving period, wherein the attendance interface 333 is provided for the caregiver to sign in or out with respect to the care service upon arrival or departure, and is related to the state of the attendance switch 140.

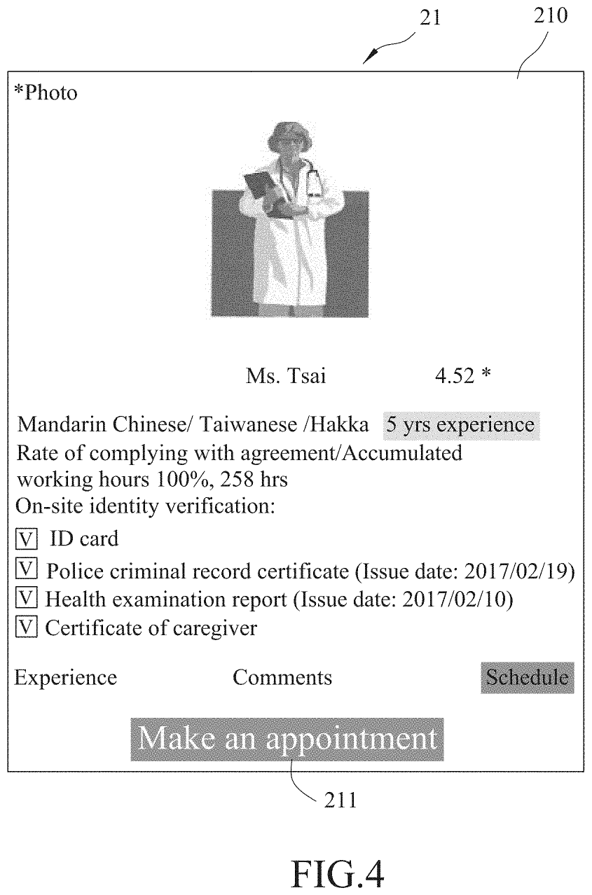

An operating procedure of the system according to the disclosure is described below. It should be noted that what are illustrated in FIGS. 4 to 13 are images of the graphical user interface rendered by execution of the app 230 or the app 350, and presented via the output unit 21 (i.e., a display device or a touchscreen) of the client terminal 2 or via the output unit 33 (i.e., a display device or a touchscreen) of the caregiver terminal 3.

To become a caregiver of the system, a person needs to operate the caregiver terminal 3 executing the registration module 351 of the app 350, and provide required data to register as a caregiver. The required data may include personal information of the caregiver (e.g., name of the caregiver, gender of the caregiver, a phone number of the caregiver and a communication address of the caregiver), a bank account, keywords that can be used in a search for the caregiver, type(s) of care service offered, a spatial range of service provision (e.g., within 500 meters, 1000 meters, 1500 meters, 2000 meters, 2500 meters, or 3000 meters of a specific location), and preferred time of service provision. Thereafter, the required data provided by the caregiver is stored in the caregiver database 11 of the server 1.

Similarly, to become a client of the system so as to request the care service for the care receiver (e.g., an elder in the family of the client), a person needs to operate the client terminal 2 executing the registration module 231 of the app 230, and provide required data to register as a client. The required data may include personal information of the client (e.g., name of the client, gender of the client, a phone number of the client and a communication address of the client), a bank account, and information related to the care receiver (e.g., name of the care receiver, gender of the care receiver, a phone number of the care receiver, and an address of a location where the care service is to be provided). Thereafter, the required data provided by the client is stored in the client database 12 of the server 1.

The client terminal 2 executing the appointment module 232 of the app 230 is configured to be operated by the client to make an appointment for the care service, and the server 1 is configured to store the appointment record 13, accordingly. Specifically speaking, the client terminal 2 executing the appointment module 232 of the app 230 is configured to be operated to access the caregiver database 11 of the server 1, and to search for caregivers that meet certain criteria inputted by the client and demanded by the care receiver (such as a specific type of care service, the care receiver's area of residence, and the time period during which the care service is required). It is noted that since the caregiver database 11 stores the required data provided by each caregiver (e.g., the keywords, the type(s) of care service offered, the spatial range of service provision, and the preferred time of service provision), the server 1 is able to provide a search result of the caregivers based on the criteria and the required data. Subsequently, the client terminal 2 executing the appointment module 232 of the app 230 is configured be operated to choose among the caregivers located through the search, and to make an appointment with the caregiver thus chosen (referred to as the chosen caregiver). Referring to FIG. 4, an appointment interface 210 provided by the output unit 21 of the client terminal 2 is illustrated. The client terminal 2 is configured to be operated by the client to select an option 211 that is marked with “Make an appointment” shown at the bottom of the appointment interface 210 so as to make an appointment with the chosen caregiver (i.e., “Ms. Tsai” shown in FIG. 4). In this embodiment, in response to the selection of the option 211 on the client terminal 2, the server 1 is configured to transmit a text message to the corresponding caregiver terminal 3 (the caregiver terminal 3 corresponding to the chosen caregiver) to notify the chosen caregiver that he/she has to respond to the client by providing a quotation in two hours.

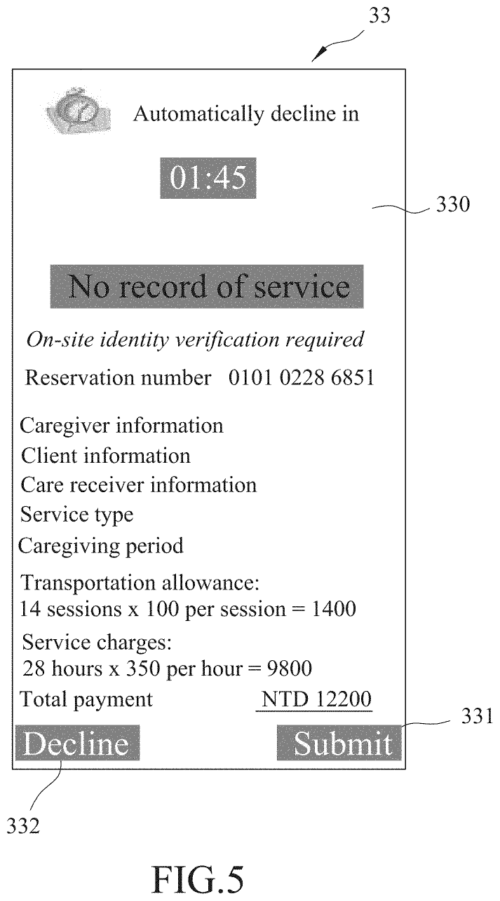

The caregiver terminal 3 executing the quotation module 352 of the app 350 is configured to be operated to provide the quotation or to decline the appointment requested by the client. As shown in FIG. 5, a quotation interface 330 provided by the output unit 33 of the caregiver terminal 3 is illustrated. To provide the quotation, the chosen caregiver has to operate the caregiver terminal 3 to provide the cost estimate (i.e., the price that the chosen caregiver will charge to provide the care service) via the quotation interface 330, and then select an option 331 that is marked with “Submit” shown at the bottom right corner of the quotation interface 330 so as to respond to the client with the quotation. Alternatively, the chosen caregiver may choose to select another option 332 that is marked with “Decline” shown at the bottom left corner of the quotation interface 330 to decline the appointment requested by the client. In this embodiment, in response to the provision of the quotation from the caregiver terminal 3, the server 1 transmits a text message to the client terminal 2 to notify the client that he/she has to approve the quotation and make payment for the care service in two hours based on the quotation provided by the chosen caregiver if he/she choose to accept the quotation. The appointment requested by the client will be automatically declined by default in case that no quotation is made by the chosen caregiver in two hours.

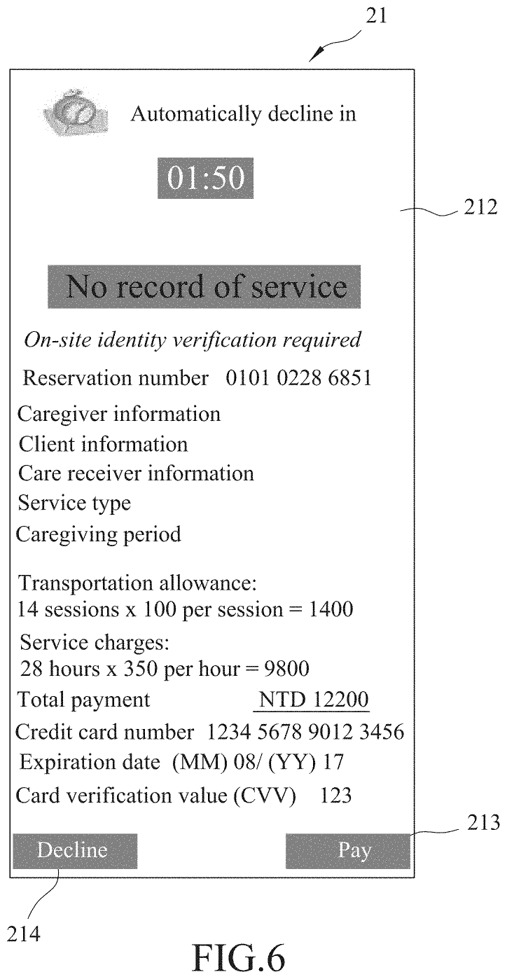

The client terminal 2 executing the payment module 233 of the app 230 is configured to be operated by the client to pay for the care service or to decline payment (i.e., to cancel the appointment). As shown in FIG. 6, a payment interface 212 provided by the output unit 21 of the client terminal 2 is illustrated. To pay for the care service, the client has to operate the client terminal 2 to provide information of a credit card to be used for the payment, e.g., a credit card number, expiration date and card verification value (CVV), and then select an option 213 that is marked with “Pay” shown at the bottom right corner of the payment interface 212 to actually fulfill the payment. Alternatively, the client may choose to select another option 214 that is marked with “Decline” shown at the bottom left corner of the payment interface 212 to cancel the appointment. The appointment will be automatically canceled by default when no response is made by the client in two hours, and in one embodiment, the server 1 may delete the appointment record 13.

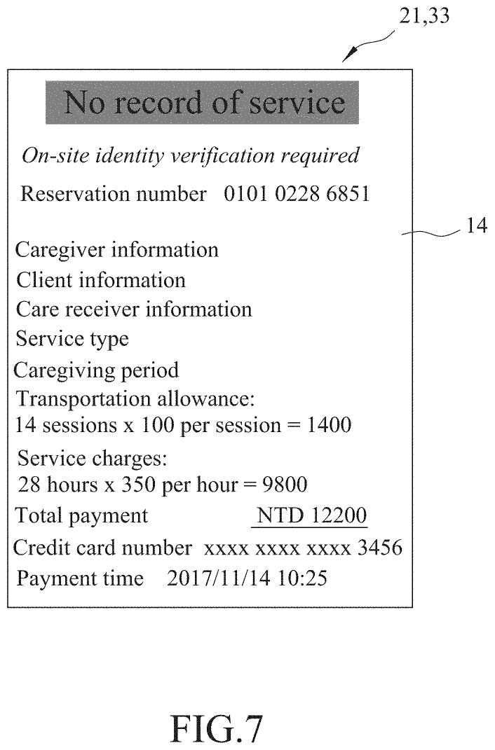

After the payment is completed, the server 1 is configured to establish the transaction record 14 based on the appointment record 13, and to store the transaction record 14. The transaction record 14 as shown in FIG. 7 is displayed on the output unit 21 of the client terminal 2 or the output unit 33 of the caregiver terminal 3, and can be checked by use of either the client terminal 2 or the caregiver terminal 3.

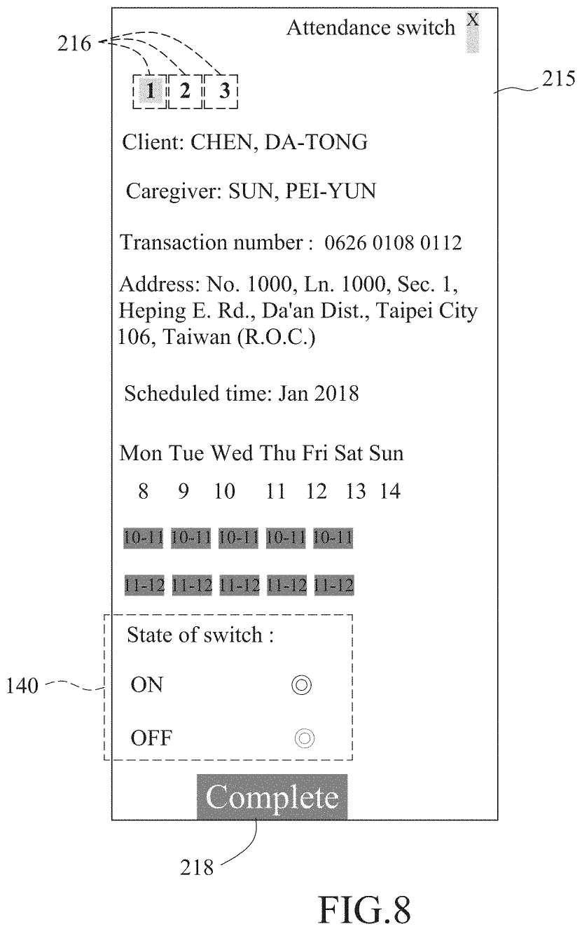

Referring to FIG. 8, the client terminal 2 executing the switch-setting module 234 of the app 230 is configured to display a switch-setting interface 215, and to be operated by the client to designate one of an “ON” state and an “OFF” state as the state of the attendance switch 140 contained in the transaction record 14 stored on the server 1. The “ON” state indicates that attendance registration (namely, the act of signing in or out) by the caregiver terminal 3 executing the signing in/out module 353 of the app 350 is allowed or enabled; the “OFF” state corresponds to attendance registration by the caregiver terminal 3 is not allowed or is disabled. The state of the attendance switch 140 contained in the transaction record 14 is designated as the “ON” state by default upon establishment of the transaction record 14. In this embodiment, after the state of the attendance switch 140 has been designated, the client terminal 2 is configured to be operated by the client to select an option 218 marked with “Complete” shown at the bottom of the switch-setting interface 215 so as to complete designating the state of the attendance switch 140.

In addition, in a scenario that a plurality of transaction records 14 corresponding to the same client (e.g., “CHEN, DA-TONG” as shown in FIG. 8) were established (e.g., a different transaction record 14 associated with a different caregiver), the transaction records 14 are enumerated based on chronological order (for instance, with respect to the start of the corresponding caregiving periods), and each of the transaction records 14 can be designated to be presented on the switch-setting interface 215 by operating the client terminal 2 to select a respective one of options 216 that are marked with numbers and that are shown to the upper left of the switch-setting interface 215. The client is able to select one of the options 216 that is marked with the corresponding number to check the state of the attendance switch 140 contained in the corresponding one of the transaction records 14.

Referring to FIG. 9, when it is determined that the state of the attendance switch 140 is the “ON” state and when it is determined that the current time is around the start of the caregiving period based on the current time determined by the timing unit 30 and the caregiving period specified in the corresponding transaction record 14, the processor 36 of the caregiver terminal 3 executing the signing in/out module 353 of the app 350 is configured to control the output unit 33 of the caregiver terminal 3 to present the attendance interface 333 that contains a registration option 334 for the caregiver to sign in or out with respect to the care service upon arrival or departure. This explains the “relation” between the attendance interface 333 and the state of the attendance switch 140 previously mentioned. In this embodiment, the processor 36 of the caregiver terminal 3 is configured to provide the attendance interface 333 to the caregiver (e.g., “SUN, PEI-YUN”, as shown in FIG. 9) when it is determined that the current time is within 30 minutes before the start of the caregiving period. For instance, the current time in FIG. 9 is “09:59”, which is within 30 minutes before the start of the caregiving period (i.e., before “10:00” when the caregiving period is 10:00-12:00 on Jan. 8, 2018), so the caregiver terminal 3 provides the attendance interface 333 via the output unit 33 for the caregiver to sign in.

When the caregiving period on one day has elapsed but the caregiver has neither signed in nor signed out, the processor 36 of the caregiver terminal 3 is configured to provide the attendance interface 333 for signing out only (i.e., signing in is not allowed) the next day. In addition, the attendance interface 333 contains a summary of the care service corresponding to the transaction record 14. When it is determined that the registration option 334 is being selected for the first time, the caregiver terminal 3 is configured to record a time instant the registration option 334 is selected as a sign-in time which represents the time when the caregiver signs in, and to transmit the sign-in time to the server 1 for storage of the sign-in time in the attendance-recording device 15 of the server 1. For example, the sign-in time is “09:59” if the registration option 334 in FIG. 9 is selected by the caregiver while the output unit 33 displays the current time as being “09:59”. Following that, the caregiver proceeds to provide the care service. When it is determined that the registration option 334 is being selected for the second time later on, the caregiver terminal 3 is configured to record another time instant the registration option 334 is selected as a sign-out time which represents the time when the caregiver signs out, and to transmit the sign-out time to the server 1 for storage of the sign-out time in the attendance-recording device 15 of the server 1.

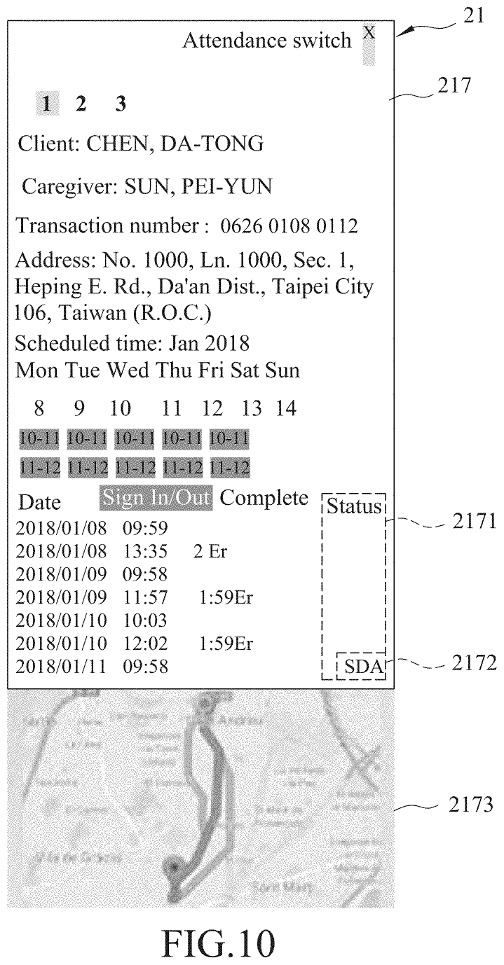

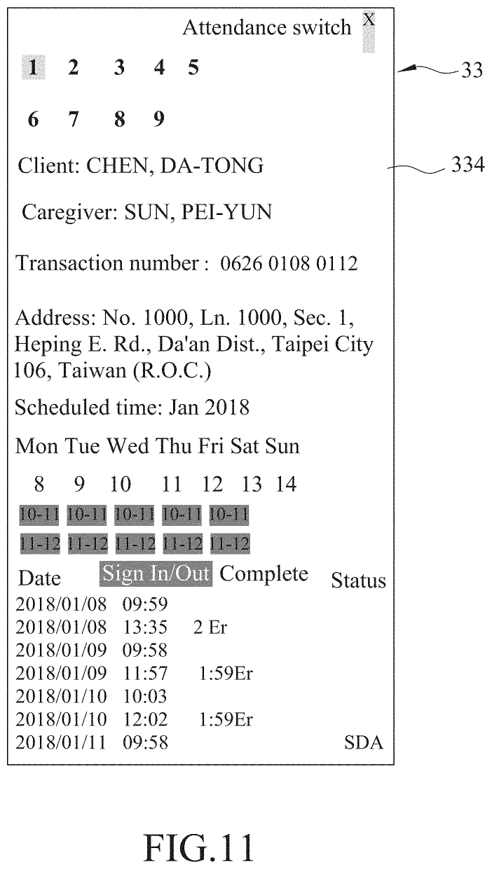

For fee calculation purposes, on the last day of every predetermined cycle (e.g., the last day of every month), the attendance-recording device 15 of the server 1 automatically collects and analyzes data related to the care service over the entire span of the predetermined cycle. For each of the predetermined cycles, the following information is obtained based on the relevant data: an accumulated number of times the care service was performed during the predetermined cycle, an accumulated number of working hours performed during the predetermined cycle, and an entry (or entries) of attendance history. Each entry of attendance history for the same care service includes a sign-in time and a sign-out time as a pair. Referring to FIGS. 10 and 11, by respectively executing the attendance-recording module 235 of the app 230 and the attendance-recording module 354 of the app 350, the client terminal 2 and the caregiver terminal 3 are configured to respectively present attendance-checking interfaces 217 and 334 that contain the entry (or entries) of attendance history corresponding to the transaction record 14 and stored in the server 1.

In this embodiment, the fee for the care service is calculated based on an hourly rate. For example, in a scenario that the hourly rate is 300 dollars, and that one caregiver ought to provide the care service for two full hours per session, if the actual working hours for one session of the care service add up to one hour and 55 minutes, then the server 1 would calculate the fee for that session as 300×(1+55/60)=575 dollars. It is noted that when the charging amount for the care service calculated by the attendance-recording device 15 of the server 1 does not match the payment amount processed previously via the client terminal 2 executing the payment module 233 of the app 230, a difference between the charging amount and the payment amount may be refunded to or charged from the client's credit card.

Moreover, referring to FIGS. 10 and 11, when it is determined that the sign-in time is later than the start of the caregiving period by 30 minutes or more, or that the actual working hours for one session of the care service exceed the duration of the caregiving period by more than one hour, or that the caregiver signed in on one day but did not sign out before two a.m. the next day, the server 1 is configured to designate a status of the entry of attendance history as an “Er” state which indicates an error. The “Er” state is marked beside the working hours of the entry of attendance history corresponding to one of the aforementioned abnormal conditions, and can be checked in the attendance-checking interface 217 (or 334) by use of the client terminal 2 (or the caregiver terminal 3).

Referring to FIG. 10, when it is determined by the client terminal 2 that the position of the caregiver terminal 3 received by the server 1 does not match the address corresponding to the location recorded in the transaction record 14 at one of the sign-in time and the sign-out time, the client terminal 2 is configured to present, along with the entry of attendance history, a map of signing in/out at different addresses (SDA) 2173 which indicates the position of the caregiver terminal 3 at the one of the sign-in time and the sign-out time and a difference between said position and the address corresponding to the location recorded in the transaction record 14 (e.g., represented as a route from said position to the location). Additionally, in this embodiment, the attendance-checking interface 217 provided by the client terminal 2 further includes an SDA block 2171. When it is determined by the client terminal 2 that the position of the caregiver terminal received by the server 1 matches the address corresponding to the location recorded in the transaction record 14 at both of the sign-in time and the sign-out time for a given session, the SDA block 2171 is left blank for that session. Otherwise, an SDA option 2172 would appear in the SDA block 2171 for that session.

Functions of the SDA block 2171, the SDA option 2172 and the map of SDA 2173 will be made clear through the following example. A caregiver is asked to run errands for the care receiver (e.g., to purchase some daily necessities or foods) at first, and then to return to the location where he/she is expected to provide the care service (referred to as a target location). Therefore, the client agrees to let the caregiver sign in outside the target location. As shown in FIG. 10, the client may select the SDA option 2172 that appears in the SDA block 2171 after the client has signed in outside the target location at 9:58 on Jan. 11, 2018. It should be noted that the SDA option 2172 appears because the position of the caregiver terminal 3 does not match the address corresponding to the target location when the caregiver 3 signed in. After the client selects the SDA option 2172, the map of SDA 2173 appears in the attendance-checking interface 217, so as to allow the client to know where the caregiver signed in. When the client selects the SDA option 2172 again, the map of SDA 2173 disappears.

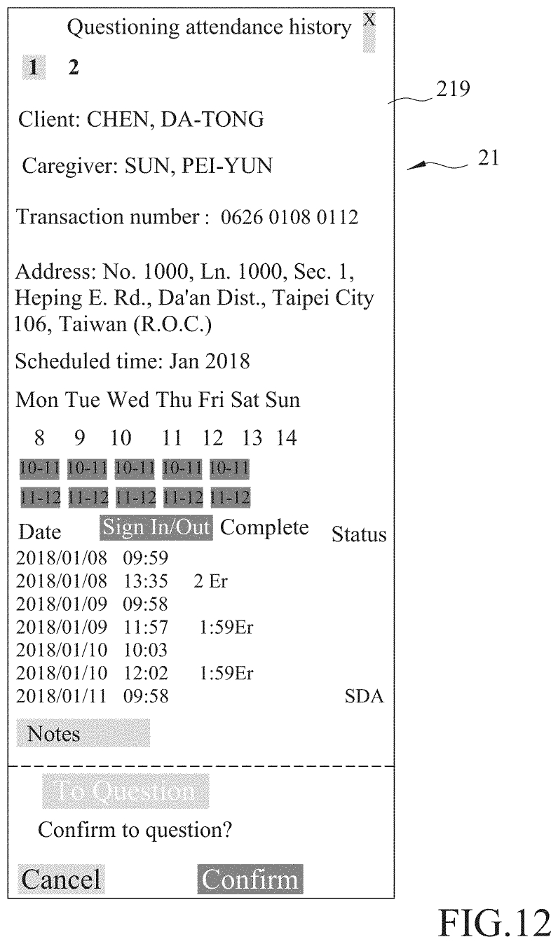

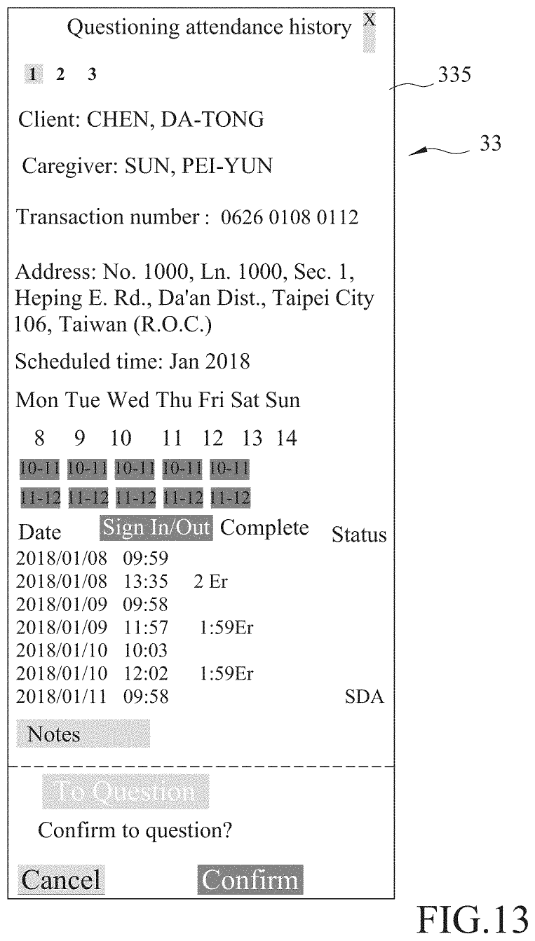

Furthermore, the server 1 executing the attendance-questioning module 16 is configured to enable, from six a.m. to twelve noon every Monday, a function of questioning the entry of attendance history corresponding to the transaction record 14. The client terminal 2 is configured to be operated by the client to question the entry of attendance history corresponding to the transaction record 14 that is stored on the server 1 when the client thinks that the entry of attendance history is questionable. Likewise, when the caregiver thinks that the entry of attendance history does not match his/her actual attendance, the caregiver terminal 3 is configured to be operated by the caregiver to question the entry of attendance history that corresponds to the transaction record 14 and that is stored on the server 1. Referring to FIGS. 12 and 13, attendance-questioning interfaces 219 and 335 respectively provided by the client terminal 2 executing the attendance-questioning module 236 of the app 230 and the caregiver terminal 3 executing the attendance-questioning module 355 of the app 350 are illustrated. In this embodiment, each of the client and the caregiver has a chance to question every entry of attendance history recorded last week via the attendance-questioning interface 219 (335).

In summary, based on demands, a client is able to operate the client terminal 2 of the system according to this disclosure to designate the state of the attendance switch 140 which is contained in the transaction record 14 stored on the server 1, so the caregiver is allowed or not allowed to utilize the caregiver terminal 3 to sign in or out for care service upon arrival or departure via the attendance interface 333. Therefore, arrival time and departure time of the caregiver may be precisely recorded in the attendance, enabling the server 1 to automatically calculate working hours of the caregiver related to the care service. In addition, the system according to the disclosure enables the client and the caregiver to utilize the function of questioning the entry of attendance history to maintain the attendance history in a fair and reasonable way.

In the description above, for the purposes of explanation, numerous specific details have been set forth in order to provide a thorough understanding of the embodiment. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A system for recording attendance of a caregiver, the caregiver being expected to provide care service at a location within a caregiving period according to an agreement on the care service made between the caregiver and a client, said system comprising:
a server configured to store a transaction record that is related to the agreement, that records the caregiving period and an address corresponding to the location, and that contains a state of an attendance switch;
a client terminal possessed by the client, capable of communicating with said server, and configured to be operated to designate the state of the attendance switch which is contained in the transaction record stored on said server; and
a caregiver terminal possessed by the caregiver, capable of communicating with said server, and including
an output unit, and
a processor that is electrically connected to said output unit and that is configured to determine whether a current time is around the start of the caregiving period, and to control said output unit to provide an attendance interface for the caregiver to sign in or out with respect to the care service upon arrival or departure when it is determined that the current time is around the start of the caregiving period, the attendance interface being related to the state of the attendance switch.

2. The system as claimed in claim 1, wherein:
said processor of said caregiver terminal is further configured to provide the attendance interface when it is determined that the current time is within 30 minutes before the start of the caregiving period.

3. The system as claimed in claim 1, wherein:
said client terminal is configured to be operated to designate one of an “ON” state and an “OFF” state as the state of the attendance switch contained in the transaction record stored on said server; and
said processor of said caregiver terminal is configured to control said output unit of said caregiver terminal to provide the attendance interface when it is determined that the state of the attendance switch is designated as the “ON” state and when it is determined that the current time is around the start of the caregiving period.

4. The system as claimed in claim 1, wherein:
the attendance interface contains a registration option; and
said caregiver terminal is configured to, when it is determined that the registration option is being selected for the first time, record a time instant the registration option is selected as a sign-in time which represents the time when the caregiver signs in, and transmit the sign-in time to said server for storage of the sign-in time in said server, and when it is determined that the registration option is being selected for the second time, record another time instant the registration option is selected as a sign-out time which represents the time when the caregiver signs out, and transmit the sign-out time to said server for storage of the sign-out time in said server.

5. The system as claimed in claim 4, wherein:
said server further records an entry of attendance history that includes the sign-in time and the sign-out time as a pair and that corresponds to the transaction record; and
said client terminal is configured to be operated to present the entry of attendance history corresponding to the transaction record and stored in said server.

6. The system as claimed in claim 5, wherein:
said caregiver terminal further includes a positioning unit that is electrically connected to said processor of said caregiver terminal and that is configured to detect a position of said caregiver terminal and to transmit the position of said caregiver terminal to said server; and
said client terminal is configured to, when it is determined by said client terminal that the position of said caregiver terminal received by said server does not match the address corresponding to the location recorded in the transaction record at one of the sign-in time and the sign-out time, present, along with the entry of attendance history, a map of signing in/out at different addresses (SDA) which indicates the position of said caregiver terminal at said one of the sign-in time and the sign-out time and a difference between said position and the address corresponding to the location recorded in the transaction record.

7. The system as claimed in claim 5, wherein said client terminal is configured to be operated to provide an attendance-questioning interface for allowing the entry of attendance history corresponding to the transaction record that is stored on said server to be questioned.

8. The system as claimed in claim 7, wherein said server is configured to enable a function of questioning the entry of attendance history corresponding to the transaction record from six a.m. to twelve noon every Monday.

9. The system as claimed in claim 4, wherein:
said server further records an entry of attendance history that includes the sign-in time and the sign-out time as a pair and that corresponds to the transaction record; and
said caregiver terminal is configured to be operated to present the entry of attendance history corresponding to the transaction record and stored in said server.

10. The system as claimed in claim 9, wherein said caregiver terminal is configured to be operated to question the entry of attendance history corresponding to the transaction record that is stored on said server.

11. The system as claimed in claim 10, wherein said server is configured to enable a function of questioning the entry of attendance history corresponding to the transaction record from six a.m. to twelve noon every Monday.
